(19) United States
(12) Patent Application Publication
(10) Pub. No.: US 2025/0366863 A1
(43) Pub. Date: Dec. 4, 2025

(54) RESECTION DEVICE

(71) Applicant: Medivation AG, Baden (CH)

(72) Inventors: Jan Stifter, Schneisingen (CH); Patricia Schmid, Zürich (CH); Roman Moor, Zürich (CH); Tobias Schwägli, Solothurn (CH); Gregor Imboden, Zürich (CH)

(73) Assignee: Medivation AG, Baden (CH)

(21) Appl. No.: 19/213,935

(22) Filed: May 20, 2025

(30) Foreign Application Priority Data
Jun. 3, 2024 (EP) 24179522.8
Jan. 28, 2025 (EP) 25154492.0

(51) Int. Cl. A61B 17/15; A61B 17/00; A61B 17/16; A61B 17/17

(52) U.S. Cl. CPC A61B 17/155; A61B 17/157; A61B 17/1764; A61B 2017/00477; A61B 2017/1602

(57) ABSTRACT

A resection device for resecting a bone of a bone arrangement without user interaction comprises a fastening element for rigid fastening to the bone arrangement and a base element rigidly connected to the fastening element. The base element includes a base element longitudinal axis. The device comprises a rotatable connecting element arranged rotatably about the base element, wherein the base element longitudinal axis forms an axis of rotation for the rotatable connecting element, and a displaceable connecting element. The displaceable connecting element performs a translation along the axis of rotation relative to the base element. The device comprises a connecting element connected to the displaceable connecting element or the rotatable connecting element via a pivot joint. The pivot joint includes a pivot joint axis of rotation. The connecting element can be rotated about the pivot joint axis of rotation. The connecting element includes an end effector for bone resection.

### CROSS-REFERENCE TO RELATED APPLICATIONS

This application claims the benefit of European patent application no. 24179522.8, filed Jun. 3, 2024, and European patent application no. 25154492.0 filed Jan. 28, 2025, the contents of both of which are hereby incorporated by reference in their entirety.

### BACKGROUND

The present invention relates to a resection device for performing resection procedures on bones. The resection device is adapted to be attached to a bone arrangement to perform a resection procedure on at least one of the bones of the bone arrangement. In particular, the resection device comprises an end effector to which a cutting tool or milling tool is attached, which is configured to follow a predetermined path, for example a predefined path, during the resection procedure. The resection device can be used for robot-assisted surgical interventions, in particular for creating precise surgical resections of bones, for example for replacing joints. In particular, the resection device can be used for knee surgery when a patient's knee joint needs to be replaced with an artificial knee joint.

### STATE OF THE ART

Resection devices are therefore used in particular in a surgical procedure in which the knee joint is replaced with an artificial knee joint, also known as total knee replacement (TKR). The main reason for this procedure is osteoarthritis, other reasons include rheumatoid arthritis, post-traumatic arthritis, or joint deformities. In the U.S. alone, this surgical procedure is performed around 790,000 times a year. During the surgery, precise incisions must be made on the femur and tibia. Typically, five incisions are made on the femur and one on the tibia using cutting blocks that guide an oscillating saw.

In order to increase the precision of these incisions and to position an implant more accurately and make the procedure easier to customize for each patient, robotic systems have been used for more than 20 years to support the surgery. For example, robotic systems include a cutting guide device that allows the surgeon to cut with an oscillating saw. Robotic systems can also consist of a large robotic arm with a hand-held end effector that allows the surgeon to perform the incision while being guided by the robotic system, as described for example in document U.S. Pat. No. 11,517,380 B2. This solution requires a navigation system that can register the position of the bones as well as the position of the end effector relative to the bones, i.e., it must cover the entire working space during the procedure. In particular, the surgeon can move within a space defined by the robotic system, with no incision being made within this space. In other words, the surgeon can perform the procedure manually within this predetermined space.

For example, a robot-assisted system according to U.S. Pat. No. 9,421,019 B2 can be used, which is attached to the bone by means of a fastening device. The cutting tool is then aligned by means of an adjustment device. However, the robot-assisted system must be guided and monitored by the surgeon for the duration of the procedure.

The position of the implant for a total knee replacement, for example, is predetermined during the planning of the surgical procedure. The predetermined position is used to define the incisions that need to be made with a resection device. Such a method is described, for example, in document U.S. Pat. No. 10,441,434 B2.

If a milling device is used for bone resection, an end effector of the milling device must fulfill many requirements in terms of size, rigidity, speed, torque and bearing, as described, for example, in document U.S. Pat. No. 9,339,345 B2. In addition, the use of a milling device for bone resection requires a suitable milling path. There are various strategies for calculating this milling path, and it is essential that the milling strategy is compatible with the system used. Document U.S. Pat. No. 8,936,596 B2 describes one way of implementation. An image of a predefined cutting pattern and an image of the bone to be resected are superimposed. The overlap between the cutting pattern and the bone is then calculated and a milling path is calculated based on this calculation result. During the calculation, care is taken to ensure that the damage to the tissue is minimized and that a thin margin remains along the circumference of the cutting plane of the bone.

With the surgical instrument system described in US20070123896 A1, bone resection is also performed by a surgeon and the surgical instrument system is positioned manually. The resection device shown in document AU 2017372744 A1 is also guided by a surgeon, for which handles are provided that are intended for manual operation of the resection device by the surgeon.

The object of the invention is to provide an autonomously operating resection device by means of which it is possible to carry out the resection without manual assistance from the surgeon and without the use of a computer-assisted navigation system, in other words automatically.

### SUMMARY OF THE INVENTION

When the term “for example” is used in the following description, this term refers to examples of embodiments and/or variants, which is not necessarily to be understood as a more preferred application of the teaching of the invention. Similarly, the terms “preferably”, “preferred” are to be understood as referring to an example from a set of embodiments and/or variants, which is not necessarily to be understood as a preferred application of the teaching of the invention. Accordingly, the terms “for example”, “preferably” or “preferred” may refer to a plurality of examples of embodiments and/or variants.

The following detailed description contains various embodiments of the resection device according to the invention. The description of a particular resection device is to be considered exemplary only. In the description and claims, the terms “including”, “comprising”, “containing” are interpreted as “including, but not limited to”.

A resection device according to the invention for resecting at least one bone of a bone arrangement without user interaction comprises a fastening element, wherein the fastening element is configured to be rigidly fastened to the bone arrangement. The resection device is configured in particular as an autonomously operating resection device. An autonomously operating resection device is understood to be a resection device that is not guided by the surgeon, at least during the performance of the resection. The resection device further comprises a base element, wherein the fastening element includes the base element, or the fastening element can be coupled to the base element in such a way that the base element forms a rigid connection with the fastening element. The base element includes a base element longitudinal axis. The resection device further comprises a rotatable connecting element rotatably arranged about the base element, wherein the rotatable connecting element is arranged rotatably about the base element longitudinal axis such that the base element longitudinal axis forms an axis of rotation for the rotatable connecting element. The rotatable connecting element includes a first connecting element drive.

The resection device further comprises a displaceable connecting element, wherein the displaceable connecting element is configured to execute a linear movement along the axis of rotation relative to the base element. The displaceable connecting element includes a second connecting element drive. A linear movement along the base element longitudinal axis relative to the base element can be performed by means of the displaceable connecting element. A linear movement is defined as a translational movement that can take place in two opposite directions. The resection device further comprises a connecting element, wherein the connecting element is connected to one of the rotatable or displaceable connecting elements via a pivot joint. The pivot joint includes a pivot joint axis of rotation, wherein the connecting element is rotatable about the pivot joint axis of rotation. The connecting element includes a third connecting element drive. The connecting element includes an end effector for bone resection. The connecting element can thus be configured as a processing device for performing the bone resection. The connecting element can form a unit with the end effector. The end effector can also be coupled to the connecting element. For example, different end effectors can be attached to the connecting element as required.

In particular, the connecting element can be configured as an articulated connecting element. In this context, the term without user interaction means that the bone is processed autonomously by the resection device.

According to an embodiment, the pivot joint axis of rotation is arranged at an angle of 60 degrees up to and including 90 degrees to the axis of rotation. In particular, the pivot joint axis of rotation can be arranged perpendicular to the axis of rotation.

A resection device according to the invention for resecting at least one bone of a bone arrangement without user interaction also comprises a fastening element, wherein the fastening element is configured to be rigidly fastened to the bone arrangement. The resection device is configured in particular as an autonomously operating resection device. An autonomously operating resection device is understood to be a resection device that is not guided by the surgeon, at least during the performance of the resection. The resection device further comprises a base element, wherein the fastening element includes the base element, or the fastening element can be coupled to the base element such that the base element forms a rigid connection with the fastening element. The base element includes a base element longitudinal axis. The resection device further comprises a rotatable connecting element rotatably arranged about the base element, wherein the rotatable connecting element is rotatably arranged about the base element longitudinal axis such that the base element longitudinal axis forms an axis of rotation for the rotatable connecting element. The rotatable connecting element includes a first connecting element drive.

The resection device further comprises a displaceable connecting element, wherein the displaceable connecting element can perform a linear movement relative to the base element along the axis of rotation. The displaceable connecting element includes a second connecting element drive. The resection device further comprises a connecting element, wherein the connecting element is connected to one of the rotatable or displaceable connecting elements via a guide rail element. The guide rail element includes a rail element axis, wherein the connecting element is displaceable along the rail element axis. The connecting element includes an end effector for bone resection. The connecting element can thus be configured as a processing device for performing bone resection. The connecting element includes a third connecting element drive. The connecting element can form a unit with the end effector. The end effector can also be coupled to the connecting element. For example, different end effectors can be attached to the connecting element as required.

According to an embodiment, the rail element axis is arranged at an angle of 60 degrees up to and including 90 degrees to the axis of rotation. In particular, the rail element axis can be arranged perpendicular to the axis of rotation.

According to an embodiment, the base element comprises a sleeve element and a core element. In particular, the base element can include an engagement element which is configured to receive a corresponding engagement element of the rotatable connecting element. According to an embodiment, the engagement element can be attached to the sleeve element. According to an embodiment, the engagement element is configured as an output gear. In particular, the output gear can be rigidly connected to the sleeve element.

According to an embodiment, the rotatable connecting element includes the corresponding engagement element and the first connecting element drive. The corresponding engagement element can be driven by means of the first connecting element drive. In particular, the corresponding engagement element can be configured as a drive gear. In particular, the first connecting element drive can comprise a drive shaft and a drive motor. According to an embodiment, the rotatable connecting element comprises a housing. The housing is configured in particular such that, when the first connecting element drive is actuated, it rotates about the axis of rotation of the base element. In particular, the housing is rotatable about the sleeve element if, according to an embodiment, such a sleeve element is provided. According to an embodiment, the housing comprises a housing shell element, a housing base element, and a housing cover element.

According to an embodiment, the first connecting element drive includes a drive housing for the drive motor. The drive housing can also be connected to the housing. In particular, the drive housing can participate in a rotational movement of the housing when the first connecting element drive is actuated. According to an embodiment, the drive shaft is rotatably mounted in the drive housing by means of the drive shaft bearing element.

In particular, the drive shaft can be set in rotation when the drive motor is started up. The drive shaft is configured in particular so that it sets the drive gear in motion. The drive gear is connected to the drive shaft in a rotationally fixed manner. The drive shaft and drive gear can also be manufactured in a single piece. According to an embodiment, the drive gear is in meshing engagement with the output gear. The output gear is connected in particular in a fixed manner to the sleeve element. The drive gear can perform a circular motion around the output gear in the manner of a planetary gear. This circular motion can be transmitted to the housing. According to an embodiment, the housing can thus rotate about the axis of rotation with the first connecting element drive. According to an embodiment, the housing includes the movable connecting element.

According to an embodiment, the displaceable connecting element is coupled to the rotatable connecting element via the housing. According to this embodiment, the housing can therefore be rotated about the axis of rotation with the first connecting element drive, with the displaceable connecting element also performing this rotational movement.

The rotatable connecting element can be rotated clockwise or counterclockwise. The direction of rotation of the rotatable connecting element can be changed. In particular, the drive shaft can be rotated clockwise or counterclockwise if the drive motor can be operated in both directions of rotation. When the resection device is used for a milling process, it must be possible to follow a specific path using the resection device. For this application, it must be possible to rotate the drive shaft in both directions. In particular, each of the three drive motors is configured to be operated in two different directions of rotation. In particular, the direction of rotation of each of the drive motors can be changed every second to follow the path. A motor controller can be provided to control and specify the direction of rotation.

According to an embodiment, the fastening element is configured for fastening to a plurality of bones of the bone arrangement, in particular at least two adjacent bones of the bone arrangement, wherein the fastening element can comprise a connector piece for forming a rigid connection. The rigid connection enables precise resection of the bone of the bone arrangement or each of the bones of the bone arrangement. According to an embodiment, a bone arrangement may also consist of only a single bone. In particular, the connector piece can be attachable to the fastening element or to the bone arrangement by means of a fastening means. According to an embodiment, the connector piece is fastened to two bones of the bone arrangement by means of corresponding fastening element. The fastening element can also be located at a different position on the bone arrangement than the connector piece. The fastening element can also be configured not to be connected to the connector piece. The fastening means can comprise at least one fastening element from the group consisting of pin elements, screw elements or clamp elements.

A soft tissue protection element can be provided to protect soft tissue from lesion by the end effector and from external objects that could impair the working space of the resection device can be provided according to an embodiment. The protection element can, for example, be configured as a protective ring or a protective bracket. The protection element can be used to ensure that no tissue or other soft tissue is damaged during assembly or operation of the end effector. In particular, the protection element can be attached to a coupling element, which is connected to the fastening element when the resection device is attached to the bone arrangement.

According to an embodiment, the fastening element includes several positions for fastening the base element. The fastening element can be made up of a plurality of parts. For example, the fastening element can comprise a plurality of fastening part elements. Each of the fastening part elements can have at least one position for fastening the base element. In particular, the positions of the fastening element can be adjustable by means of an adjustable coupling element.

According to an embodiment, the fastening element is configured as an intramedullary rod or a modified intramedullary rod. The fastening element can also comprise a fastening means from the group consisting of a bone clamp, a bone nail, a bone screw, and a holder fastened with pin elements.

According to an embodiment, the tool is configured as an element from the group consisting of a milling device, a sawing device, and a drilling device. In particular, the tool can comprise a ball end mill.

According to each of the embodiments, the rotatable connecting element, the displaceable connecting element, and the connecting element can be driven independently of one another by means of corresponding connecting element drives. In particular, the rotatable connecting element can be driven by a first connecting element drive, the displaceable connecting element can be driven by a second connecting element drive and the connecting element can be driven by a third connecting element drive. In particular, at least one of the connecting element drives is housed in a compact block that can be removed from the housing. For example, the arrangement of motors in a compact, removable block facilitates their maintenance and assembly.

According to an embodiment, the first connecting element drive and the second connecting element drive are arranged in a common housing. According to an embodiment, the first connecting element drive is used to rotate the housing of the resection device about the base element longitudinal axis, with the base element longitudinal axis of forming the axis of rotation. According to this embodiment, the rotatable connecting element is formed by the housing. According to this embodiment, the displaceable connecting element is attached to the housing in such a way that when the rotatable connecting element rotates, the displaceable connecting element is also affected by this rotation.

The translational movement of the displaceable connecting element can take place simultaneously or with a time delay in relation to the rotation of the rotatable connecting element. It is therefore possible for the rotatable and the displaceable connecting element to be actuated simultaneously. It is also possible that only a rotation of the rotatable connecting element takes place at a first point in time and only the displacement of the displaceable connecting element takes place at a second point in time. The first point in time can therefore differ from the second point in time. The first and second points in time can also coincide if the first connecting element drive and the second connecting element drive are in operation at the same time.

The rotation of the connecting element can also take place at the same time or with a time delay in relation to the rotation of the rotatable connecting element or the translational movement of the displaceable connecting element. It is also possible that only a rotation of the rotatable connecting element takes place at a first point in time and only the displacement of the displaceable connecting element takes place at a second point in time and only the rotation or displacement of the connecting element takes place at a third point in time. The first point in time can therefore differ from at least one of the second or third points in time. The first, second and third points in time can also coincide if the first connecting element drive and the second connecting element drive and the third connecting element drive are in operation at the same time.

The rotatable connecting element, the displaceable connecting element and the connecting element can be driven independently of one another by means of corresponding connecting element drives. In particular, the rotatable connecting element can be driven by a first connecting element drive, the displaceable connecting element can be driven by a second connecting element drive and the connecting element can be driven by a third connecting element drive. In particular, at least one of the connecting element drives is accommodated in a compact block that can be removed from the housing. If the housing is formed by the rotatable connecting element, the displaceable connecting element can be arranged on the housing. For example, according to an embodiment, the arrangement of the of at least two of the first, second or third connecting element drives in or on a compact, removable block facilitates maintenance and assembly of the resection device.

According to an embodiment, the first connecting element drive and the second connecting element drive are arranged in or on a common housing. By means of the first connecting element drive, the housing of the resection device can be rotated about the base element longitudinal axis, with the base element longitudinal axis forming the axis of rotation. The rotatable connecting element can be formed by the housing. According to an embodiment, the displaceable connecting element is attached to the housing in such a way that when the rotatable connecting element is rotated, the displaceable connecting element is also affected by this rotation.

In particular, the resection device according to each of the embodiments has a modular structure. The modular structure makes it easier to implement a sterile concept. For example, one part of the resection device can be configured to be sterile, while another part of the resection device can be configured to be non-sterile. At least part of the resection device can be covered with a sterile drape, which can be configured as a sterile cover.

The invention thus relates to a robot-assisted resection device for performing active, i.e., autonomously performed, resections of at least one of the bones of the bone arrangement, e.g., the distal femur in a joint replacement operation. The resection device comprises a fastening element, a base element which is rigidly connected to the fastening element, and a plurality of movable members which are attached as connecting elements to the base element or to one another in a rotatable or displaceable manner, wherein the last movable member is configured as an end effector. The end effector can include a bone resection tool, for example a milling device or a sawing device. The fastening device can, for example, be configured as at least one element from the group consisting of an intramedullary rod, a modified intramedullary rod, a bone nail, a bone screw, and a bone clamp.

According to the invention, the end effector autonomously follows a predefined path without the need for human intervention and resects the at least one of the bones of the bone arrangement accordingly. The resection can be performed on the bone of the bone arrangement to which the resection device is attached or on an adjacent bone, for example when the resection device is attached to an intramedullary rod in the femur and both femur and tibia are resected. The fastening element of the resection device does not necessarily have to be repositioned, which can result in a considerable reduction in the duration of the procedure. The two bones of the bone arrangement can be rigidly connected to each other by a connector piece.

One application for the resection device according to the invention can be the resection of the femur and tibia for a total knee replacement.

Further advantages of the resection device according to the invention are that it is attached to the bone arrangement, has a low dead weight and performs active, i.e., autonomous, or automatic, processing of the at least one bone of the bone arrangement. By attaching the resection device to the bone arrangement, the processing time required for the procedure can be reduced, in particular because the setup can be simplified, and in particular the time until the resection device is put into operation can be shortened, since registration of the resection device in relation to the patient can be dispensed with.

According to the invention, the resection device is fixed to the patient in an unchangeable position. Because the step of registering the resection device can be omitted, the processing time can be reduced, i.e., the duration of the procedure can be shortened, which results in less stress for the patient. With the one-time alignment of the resection device during assembly, its position is already fixed at the beginning of the procedure and can no longer change due to the rigid fixation, so that additional working time for subsequent adjustments on the patient can also be omitted. The design of the resection device according to the invention is particularly compact since the design of the resection device is aligned with the bone axis. Due to the compact design, a navigation system is only required for a comparatively small working space. In addition, the small working space can be processed more precisely. As the resection device has a more compact design than previously known solutions, it can be manufactured more cost-effectively.

If the end effector also includes a milling device, the procedure can be performed more gently. According to a large number of research results, milling has proven to be gentler for the resection of the at least one bone of the bone arrangement. In particular, the milling process generates less heat. The reduced exposure to heat causes less damage to the bone. The resection device also works with increased precision because it is fixed directly to the bone arrangement. As the resection device forms a single compact system that remains rigidly connected to the patient, it does not need to be aligned before the procedure. In contrast to conventional robotic systems which are not connected to the patient, and which have to be constantly carried along with the patient, the resection device according to the invention is already fixed to the patient. This means that the procedure can be performed on the patient more cost-effectively, fewer components are required and handling during the procedure is simpler.

Furthermore, with the resection device according to the invention, an arrangement of three degrees of freedom for the operation of the end effector and a tool coupled to the end effector can be realized by means of the resection device itself, without the need for further components.

### BRIEF DESCRIPTION OF THE DRAWINGS

The resection device according to the invention is illustrated below with reference to the drawings, wherein

FIG. 1 shows a lateral view of a resection device according to a first embodiment, which is attached to a bone arrangement,

FIG. 2 shows a side view of the resection device according to FIG. 1,

FIG. 3 shows a frontal view of the resection device according to FIG. 1,

FIG. 4 shows a perspective view of the resection device according to FIG. 1,

FIG. 5 shows a further perspective view of the resection device according to FIG. 1,

FIG. 6 shows a further perspective view of the resection device according to FIG. 1,

FIG. 7 shows a perspective view of a variant of the resection device according to FIG. 1,

FIG. 8 shows a further perspective view of the resection device according to FIG. 1,

FIG. 9 shows a perspective view of a variant of the resection device according to FIG. 1,

FIG. 10 shows a perspective view of the resection device and a connector piece according to a first variant for creating a rigid connection between femur and tibia,

FIG. 11 shows a perspective view of a connector piece according to a second variant for creating a rigid connection between femur and tibia,

FIG. 12 shows a perspective view of a connector piece according to a third variant for creating a rigid connection between femur and tibia,

FIG. 13 shows a perspective view of a connector piece according to a fourth variant for creating a rigid connection between femur and tibia,

FIG. 14 shows a perspective view of a connector piece according to a fifth variant for creating a rigid connection between femur and tibia,

FIG. 15 shows a side view of a resection device according to a second embodiment,

FIG. 16 shows a view of a resection device with a soft tissue protection element,

FIG. 17 shows a view of a resection device with a variant of the fastening element,

FIG. 18 shows a view of a resection device according to FIG. 1 with a partially omitted housing,

FIG. 19a shows a first view of a resection device according to a third embodiment,

FIG. 19b shows a section through the resection device according to FIG. 19a along the section plane B-B,

FIG. 20a shows a second view of a resection device according to the third embodiment,

FIG. 20b shows a section through the resection device according to FIG. 20a along the section plane C-C.

### DETAILED DESCRIPTION

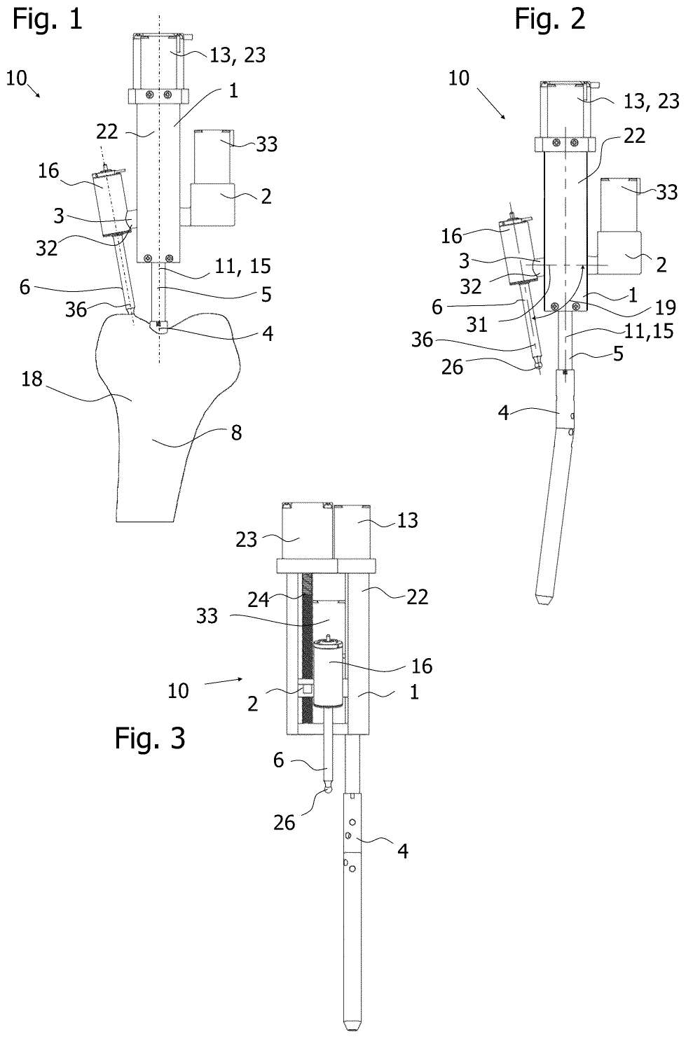

FIG. 1 shows a lateral view of a resection device 10 according to a first embodiment of the invention, which is attached to a bone arrangement 8, FIG. 2 shows a side view of the resection device 10 according to FIG. 1 and FIG. 3 shows a frontal view of the resection device according to FIG. 1. The resection device 10 for resection of at least one bone of the bone arrangement 8 without user interaction comprises a fastening element 4, wherein the fastening element 4 is configured to be rigidly attached to the bone arrangement 8. The resection device 10 further comprises a base element 5, wherein the fastening element includes the base element 5 or the fastening element 4 can be coupled to the base element 5 in such a way that the base element 5 forms a rigid connection with the fastening element 4. The base element 5 comprises a base element longitudinal axis 15. The resection device 10 further comprises a rotatable connecting element 1 which is arranged rotatably about the base element 5, wherein the rotatable connecting element 1 is arranged rotatably about the longitudinal axis 15 of the base element, so that the base element longitudinal axis 15 forms an axis of rotation 11 for the rotatable connecting element 1. The rotatable connecting element can comprise a housing. The resection device 10 further comprises a displaceable connecting element 2, wherein the displaceable connecting element 2 can perform a linear movement along the axis of rotation 11 relative to the base element 5.

The resection device 10 further comprises a connecting element 3, wherein the connecting element 3 is connected to one of the rotatable or displaceable connecting elements 1, 2 via a pivot joint 32. The connecting element 3 is configured as a processing device for performing the bone resection. The pivot joint 32 includes a pivot joint axis of rotation 31, wherein the connecting element 3 is rotatable about the pivot joint axis of rotation 31, wherein the pivot joint axis of rotation 31 is arranged perpendicular to the axis of rotation 11 according to this embodiment. The connecting element 3 includes an end effector 6 for the bone resection. The connecting element 3 can form a unit with the end effector 6. The end effector 6 can also be coupled to the connecting element 3. For example, different end effectors 6 can be attached to the connecting element 3 as required. Of course, according to an embodiment not shown, the connecting element provided with reference sign 2 can be configured as the rotatable connecting element and the connecting element provided with reference sign 1 can be configured as the displaceable connecting element.

The end effector 6 includes the tool 26, in particular a bone resection tool, which can, for example, be configured as a milling device or a sawing element. The end effector 6 can include an end effector rotation axis 36 about which the tool 26 may rotate. The end effector rotation axis 36 can coincide with the longitudinal axis of the end effector 6. In addition, the end effector can have an end effector drive 16.

The rotatable connecting element 1, the displaceable connecting element 2 and the connecting element 3 can be driven independently of one another by means of corresponding connecting element drives 13, 23, 33. In particular, the rotatable connecting element 1 can be driven by a first connecting element drive 13, the displaceable connecting element 2 can be driven by a second connecting element drive 23 and the connecting element 3 can be driven by a third connecting element drive 33. In particular, at least one of the connecting element drives 13, 23, 33 is accommodated in a compact block that can be removed from the housing. According to the present embodiment, the rotatable connecting element 1 forms a housing 22 and the displaceable connecting element 2 is arranged on the housing 22. According to an embodiment not shown, a common housing 22 is formed by the rotatable connecting element 1 and the displaceable connecting element 2.

According to this embodiment, the first connecting element drive 13 and the second connecting element drive 23 are arranged in or on a common housing 22. The first connecting element drive 1 is used to rotate the housing 22 of the resection device 10 about the longitudinal axis 15 of the base element, with the base element longitudinal axis 15 forming the axis of rotation 11. According to this embodiment, the rotatable connecting element 1 is formed by the housing 22. According to this embodiment, the displaceable connecting element 2 is attached to the housing 22 in such a way that when the rotatable connecting element 1 is rotated, the displaceable connecting element 2 is also affected by this rotation. The translational movement of the displaceable connecting element 2 can take place simultaneously or with a time delay in relation to the rotation of the rotatable connecting element 1. It is therefore possible for the rotatable and the displaceable connecting element 1, 2 to be actuated simultaneously.

It is also possible that at a first point in time only the rotation of the rotatable connecting element 1 takes place and at a second point in time only the displacement of the displaceable connecting element 2 takes place. The first point in time can therefore differ from the second point in time. The first and second points in time can also coincide if the first connecting element drive 13 and the second connecting element drive 23 are in operation at the same time. The rotation of the connecting element 3 can also take place at the same time or with a time delay in relation to the rotation of the rotatable connecting element 1 or the translational movement of the displaceable connecting element 2. It is also possible that at a first point in time only a rotation of the rotatable connecting element 1 takes place and at a second point in time only the displacement of the displaceable connecting element 2 takes place and at a third point in time only the rotation or displacement of the connecting element 3 takes place. The first point in time can therefore differ from at least one of the second or third points in time. The first, second and third points in time can also coincide if the first connecting element drive 13, the second connecting element drive 23 and the third connecting element drive 33 are in operation at the same time.

The fastening element 4 can, for example, be configured as an intramedullary (IM) rod or as another fixation. The fixation can be attached to a bone arrangement 8, for example a femur 18, as shown in FIG. 1. The bone arrangement 8 may consist of a single bone or comprise a plurality of bones.

According to this embodiment, the resection device has at least three degrees of freedom (DOF). The first DOF comprises a rotation of the rotatable connecting element 1 about the axis of rotation 11, which corresponds to the base element longitudinal axis 15 of the base element 5 or, according to an embodiment not shown in the drawing, runs parallel to the base element longitudinal axis 15. The second DOF comprises a translational movement of the displaceable connecting element 2 in the direction of the axis of rotation 11 of the rotatable connecting element 1. According to the present embodiment, the third DOF comprises a rotation of the connecting element 3 about a pivot joint axis of rotation 31. The pivot joint axis of rotation can in particular be perpendicular to the axis of rotation 11 of the rotatable connecting element 1, it can also, for example, be arranged at an angle of 60 degrees up to and including 90 degrees to the axis of rotation 11.

FIG. 2 shows a side view of the resection device 10 according to FIG. 1. Identical or similarly acting components bear the same reference numerals as in FIG. 1. For these components, please refer to the description in FIG. 1. Similarly, reference is also made to the description of FIG. 1 for the same or similarly acting components in FIGS. 2-10 and 16-18.

As an exemplary embodiment, FIG. 2 shows that the end effector rotation axis 36 of the end effector 6 is arranged at an angle of inclination 19 with respect to the pivot joint axis of rotation 31. If the end effector rotation axis 36 includes an angle of inclination 19 with respect to the pivot joint axis of rotation 31, a larger working surface can be machined using the end effector 6. In particular, portions of the working surface that are located on the bone arrangement 8 below the housing 22 in the graphic representation can also be machined. The angle of inclination 19 can be 60 up to and including 90 degrees. The angle of inclination 19 shown in FIG. 2 is only to be regarded as an exemplary embodiment of a large number of possible angles of inclination. Different angles of inclination can be realized by different designs of the connecting element 3 if required.

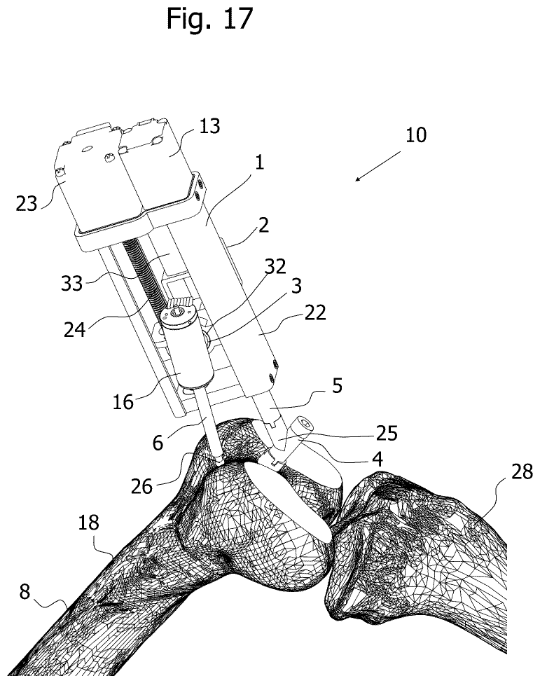

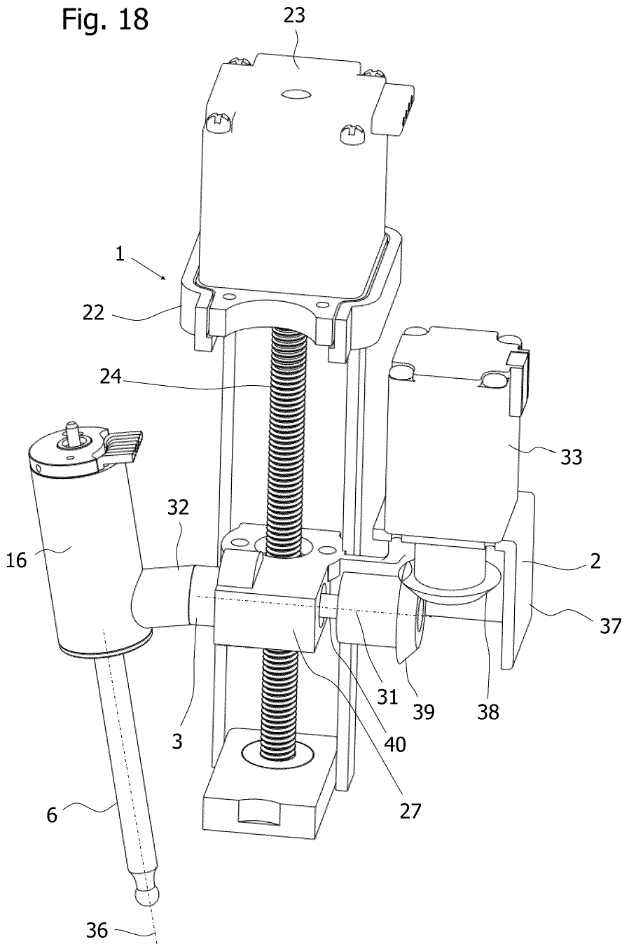

In particular, the fastening element 4 can include several positions for fastening the base element 5 so that the resection device 10 can already be positioned in the best possible way in relation to the work space. If necessary, the positions can be set by means of an adjustable coupling element, which is shown in FIG. 17, for example.

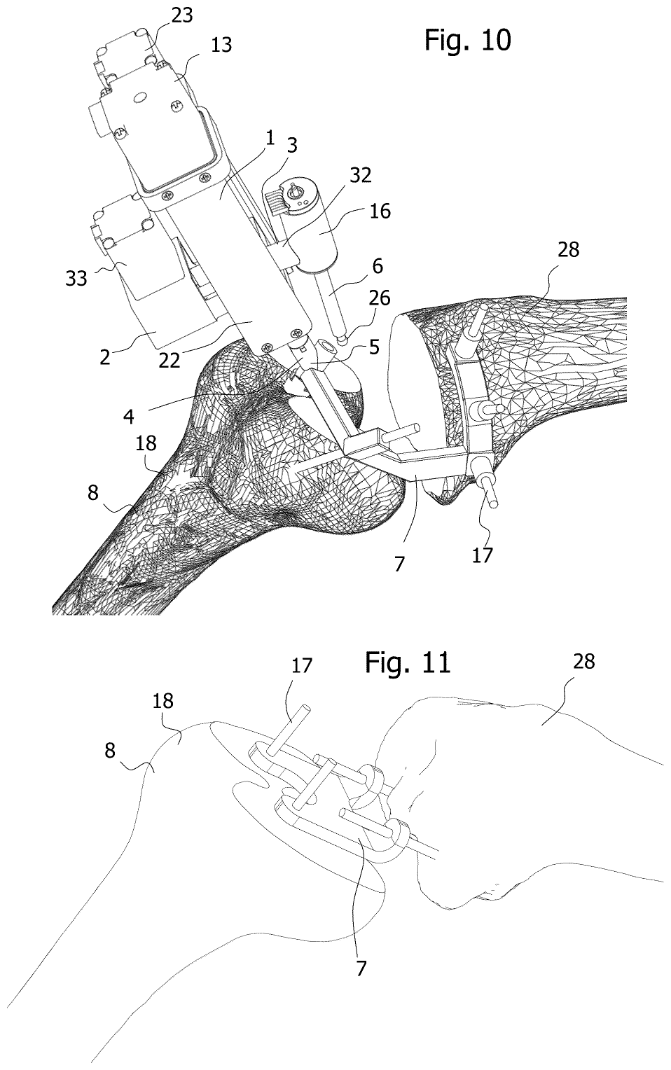

The fastening element 4 can be configured as an element from the group consisting of an intramedullary rod, a modified intramedullary rod, a bone nail, a bone screw, and a bone clamp. The fastening element 4 can include a connector piece 7 or can be rigidly coupled to a connector piece 7. Examples of embodiments of fastening elements and different variants of connector pieces 7 are shown in FIGS. 10 to 14.

The tool 26 can comprise an element from the group consisting of a milling device, a sawing device, and a drilling device. In particular, the tool 26 may comprise a ball end mill.

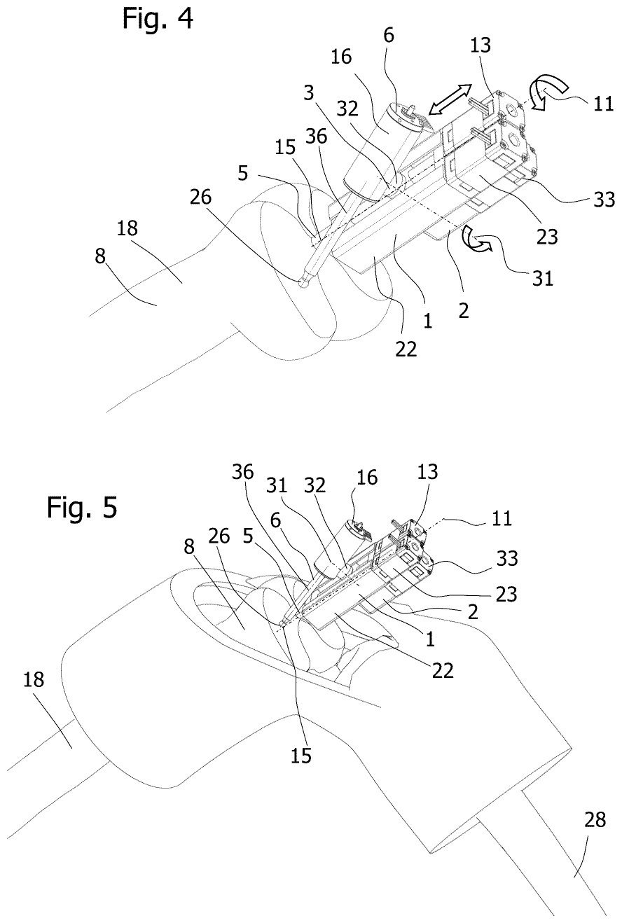

FIG. 4 shows a perspective view of the resection device 10 according to FIG. 1, which is attached to the femur 18 on a fastening element, which is configured as an intramedullary rod. The distal resection of the femur 18 for a total knee replacement has already been performed.

FIG. 5 shows a perspective view of the resection device 10 according to FIG. 1, which is attached to the femur 18 on a fastening element configured as an intramedullary rod, with a rough model of the soft tissue surrounding the resection space. The distal resection of the femur 18 for a total knee replacement has already been performed.

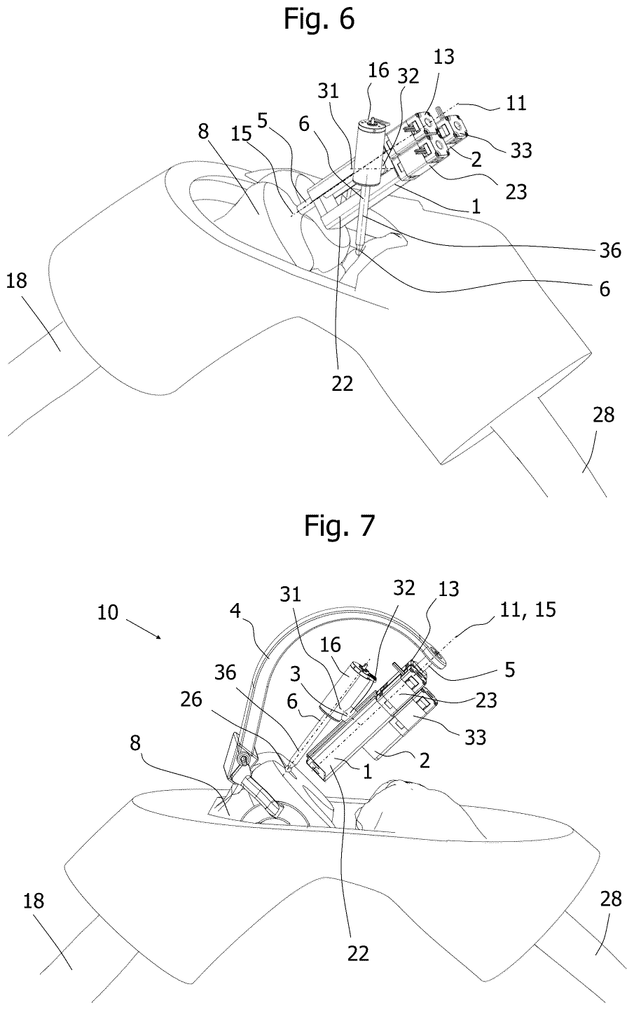

FIG. 6 shows a perspective view of the resection device 10 according to FIG. 1, which is attached to the femur 18 on a fastening element, which is configured as an intramedullary rod, with a rough model of the soft tissue surrounding the resection space. The distal resection of the femur 18 for a total knee replacement has already been performed and the resection device 10 performs the resection of the tibia 28.

FIG. 7 shows a perspective view of the resection device 10 according to FIG. 1 according to a variant. The fastening element 4 of the resection device 10 according to FIG. 7 is configured as a specially formed bone clamp on the femur 18. FIG. 7 also shows a rough model of the surrounding soft tissue. The distal resection of the femur 18 for a total knee replacement has already been performed.

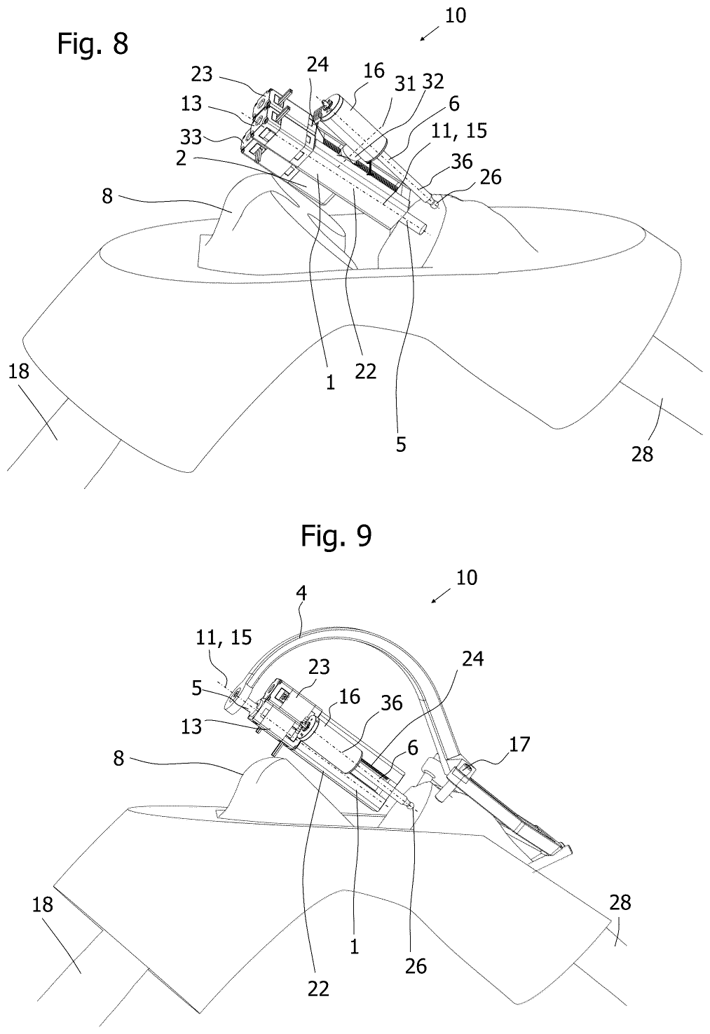

FIG. 8 shows a perspective view of the resection device 10 according to FIG. 1, which is attached to the tibia 28 on a fastening element in the form of an intramedullary rod, with a rough model of the soft tissue surrounding the resection space. The distal resection of the femur 18 and the proximal resection of the tibia 28 for a total knee replacement have already been performed.

FIG. 9 shows a perspective view of a variant of the resection device 10 according to FIG. 1, the fixation element 4 is configured in the form of a special holder, which is attached to the tibia 28 with bone nails 17, with a rough model of the of the soft tissue surrounding the resection space. The distal resection of the femur 18 and the proximal resection of the tibia 28 for a total knee replacement have already been performed.

FIG. 10 shows a perspective view of the resection device 10 according to FIG. 1, the femur 18 and the tibia 28 with a possible configuration of a fastening element 4 for establishing a rigid connection between the femur 18 and the tibia 28. The fastening element 4 comprises a connector piece 7 according to a first variant. According to the present embodiment, the connector piece 7 is attached to the fastening element 4 of the femur 18 and fixed to the tibia 28 with bone nails 17. When the resection device 10 is used to resect another bone of the bone arrangement 8 that does not correspond to the bone of the bone arrangement 8 to which it is attached, for example, to resect the tibia 28 while it is attached to the femur 18, as shown in FIG. 6. The femur 18 and tibia 28 are rigidly connected to each other by means of the connector piece 7. The connector piece 7 can be configured as a plate with bone nails 17 for attachment to the bone arrangement, as shown in FIG. 11. Other possibilities include the use of bone clamps 9 and combinations of bone nails 17 and bone clamps 9 to attach the connector piece 7 to the bone arrangement 8, as shown in FIG. 11 to FIG. 14. Instead of bone nails, bone pins or bone screws can also be used according to any of the embodiments.

FIG. 11 shows a perspective view of the femur 18 and the tibia 28 with a possible configuration of a connector piece 7 according to a second variant for establishing a rigid connection between the femur 18 and the tibia 28. According to this embodiment, the connector piece 7 is configured as a plate which is attached to bone nails 17 at the distal end of the femur 18 and at the proximal end of the tibia 28.

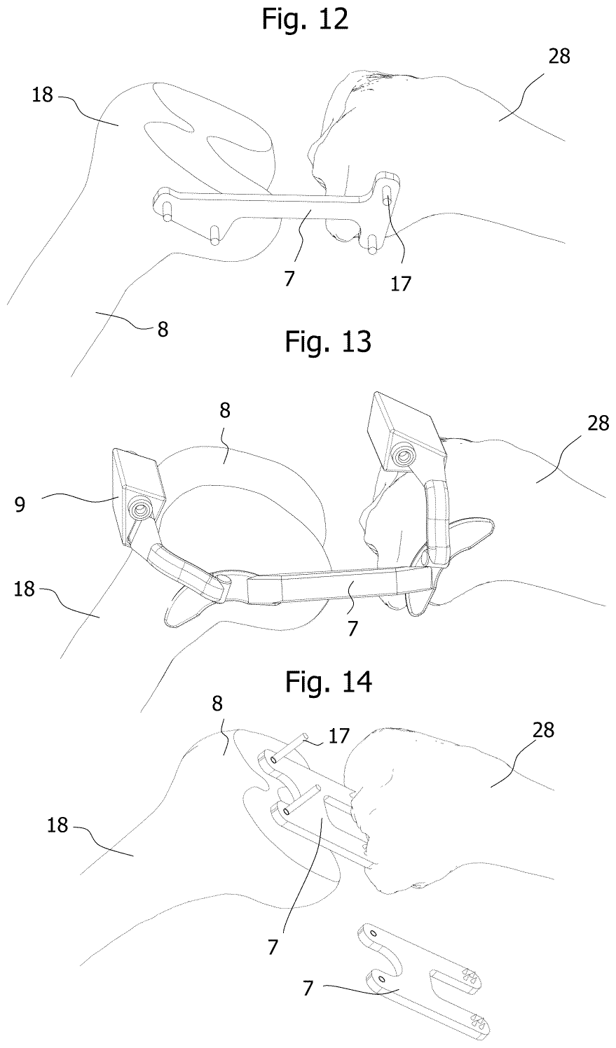

FIG. 12 shows a perspective view of the femur 18 and the tibia 28 with a possible configuration of a connector piece 7 according to a third variant for establishing a rigid connection between the femur 18 and the tibia 28. The connector piece 7 is fixed with bone nails 17 from the medial side in the femur and tibia.

FIG. 13 shows a perspective view of femur 18 and tibia 28 with a possible configuration of a connector piece 7 according to a fourth variant for establishing a rigid connection between femur 18 and tibia 28. The connector piece is attached directly to the femur and tibia using bone clamps 9.

FIG. 14 shows a perspective view of femur 18 and tibia 28 with a possible configuration of a connector piece 7 according to a fifth variant for establishing a rigid connection between femur 18 and tibia 28. The connector piece 7 according to this embodiment is configured as a plate which is fixed with bone nails 17 at the distal end of femur 18 and a bone clamp at the proximal end of tibia 28.

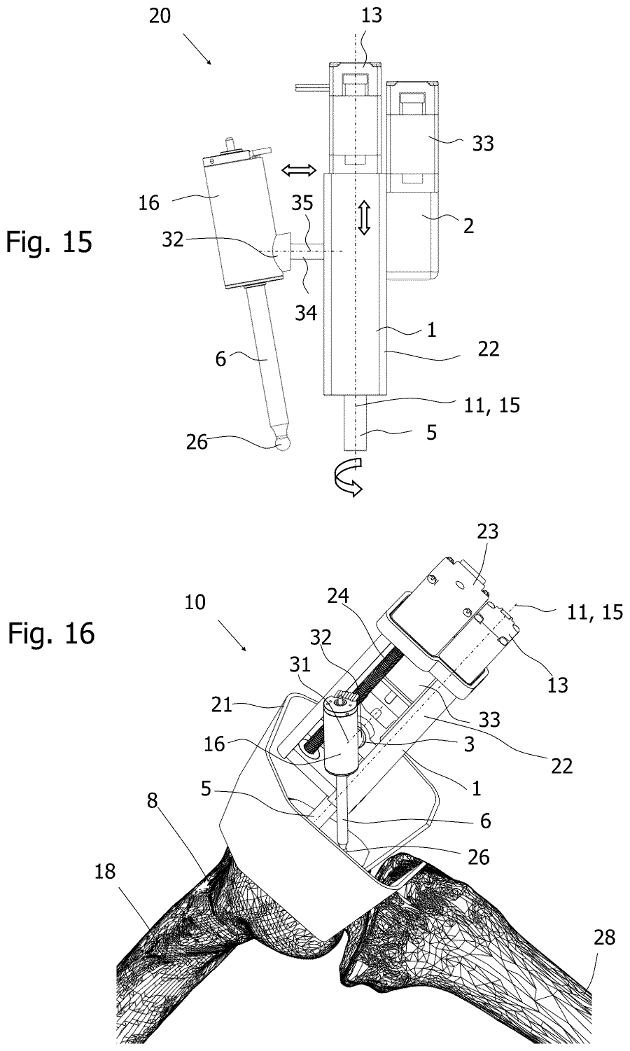

FIG. 15 shows a side view of a resection device 20 according to a second embodiment. According to the present embodiment, the movement sequences correspond to the first and second degrees of freedom of the first embodiment. For this embodiment, the same reference signs are used for identical or similarly acting elements as in the preceding embodiments. According to this embodiment, the third degree of freedom comprises a translation perpendicular to the axis of rotation of the first degree of freedom.

The resection device 20 for resection of at least one bone of a bone arrangement 8 without user interaction according to the embodiment shown in FIG. 15 comprises, like the resection device 10 shown in FIG. 1, a fastening element 4, wherein the fastening element 4 is configured to be rigidly fastened to the bone arrangement. The fastening element 4 is omitted in the present embodiment, please refer in particular to FIG. 1 to FIG. 3. In addition, the resection device 20 comprises a base element 5, wherein the fastening element 4 includes the base element 5 or the fastening element 4 can be coupled to the base element 5 in such a way that the base element 5 forms a rigid connection with the fastening element 4. The base element 5 comprises a base element longitudinal axis 15. The resection device 20 comprises a rotatable connecting element 1 which is arranged rotatably about the base element 5, wherein the rotatable connecting element 1 is arranged rotatably about the base element longitudinal axis 15, so that the base element longitudinal axis 15 forms an axis of rotation 11 for the rotatable connecting element 1.

The resection device 20 further comprises a displaceable connecting element 2, wherein the displaceable connecting element 2 can perform a linear movement along the axis of rotation 11 relative to the base element 5. The resection device 20 further comprises a connecting element 3, wherein the connecting element 3 is connected to the displaceable connecting element 2 via a guide rail element 34. The guide rail element 34 includes a rail element axis 35, wherein the connecting element 3 is displaceable along the rail element axis 35. According to this embodiment, the rail element axis 35 is arranged perpendicular to the axis of rotation 11. The connecting element 3 includes an end effector 6 for bone resection.

In particular, the fastening element 4 can include several positions for fastening the base element 5 so that the resection device can already be positioned in the best possible way in relation to the work space or a plurality of work spaces. If necessary, the positions can be set by means of an adjustable coupling element, which is shown in FIG. 17, for example.

The fastening element 4 can be configured as a fastening means from the group consisting of an intramedullary rod, a modified intramedullary rod, a bone nail, a bone clamp, a bone screw, and a holder fastened with pin elements. The fastening element 4 can include a connector piece 7 or can be rigidly coupled to a connector piece 7. FIGS. 10 to 14 show examples of fastening elements and any associated connector pieces 7.

The tool 26 can comprise an element from the group consisting of a milling device, a sawing device, and a drilling device. In particular, the tool 26 can comprise a ball end mill.

FIG. 16 shows a view of a resection device 10 with a soft tissue protection element 21. The soft tissue protection element 21 has the function of protecting soft tissue from the end effector 6 and/or from external objects which may impair the working space of the resection device. In particular, the soft tissue protection element 21 can be configured as a tissue protector. The soft tissue protection element 21 can be attached to at least one of the bone arrangement 8, to the fastening element 4, to the base element 5 or to the connector piece 7.

FIG. 17 shows a view of a resection device 10 comprising a variant of the fastening element 4, which includes several positions for fastening the base element 5. According to the present embodiment, the positions can be adjusted by means of an adjustable coupling element 25.

FIG. 18 shows a detail of a resection device 10, in which the housing 22 and the rotatable connecting element 1, which is arranged rotatably about the base element 5, are partially omitted. The resection device 10 comprises a displaceable connecting element 2, wherein the displaceable connecting element 2 can perform a linear movement along the axis of rotation 11. The axis of rotation 11 is not visible in this illustration, it runs parallel to the center axis of a spindle element 24, which is a component of the displaceable connecting element 2. The resection device 10 further comprises a connecting element 3, wherein the connecting element 3 is connected to one of the rotatable or displaceable connecting elements 1, 2 via a pivot joint 32. The connecting element 3 is configured as a processing device for performing the bone resection. The pivot joint 32 includes a pivot joint axis of rotation 31, wherein the connecting element 3 is rotatable about the pivot joint axis of rotation 31, wherein the pivot joint axis of rotation 31 according to this embodiment is arranged at an angle of inclination to the axis of rotation 11, which can be in the range from 60 degrees up to and including 90 degrees. The connecting element 3 includes an end effector 6 for bone resection. The connecting element 3 can form a unit with the end effector 6. The end effector 6 can also be coupled to the connecting element 3. For example, different end effectors 6 can be attached to the connecting element 3 as required.

The end effector 6 includes the tool 26, in particular a bone resection tool, which can, for example, be a milling device or a sawing element. The end effector 6 can include an end effector rotation axis 36 about which the tool 26 may rotate. The end effector rotation axis 36 can coincide with the longitudinal axis of the end effector 6. In addition, the end effector can have an end effector drive 16.

The rotatable connecting element 1, the displaceable connecting element 2 and the connecting element 3 can be driven independently of one another by means of corresponding connecting element drives 13, 23, 33. In particular, the rotatable connecting element 1 can be driven by a first connecting element drive 13, not shown, which is attached to the housing 22 only partially shown. The displaceable connecting element 2 is driven by a second connecting element drive 23. The connecting element 3 is driven by a third connecting element drive 33. According to the present embodiment, the rotatable connecting element 1 forms a housing 22. The displaceable connecting element 2 is arranged in the housing 22.

According to this embodiment, the displaceable connecting element 2 comprises the second connecting element drive 23, the spindle element 24 driven by the second connecting element drive 23 and a slide element 27 displaceable with the spindle element 24. The slide element 27 comprises a holder 37 for the third connecting element drive 33. According to this embodiment, the slide element 27 and the holder 37 are configured as a single component. However, according to an embodiment not shown, it is also possible for the holder and the slide element to be configured as two or more components which are, however, rigidly connected to one another, for example with a screw connection, a welded connection or another connecting means. For the function of the connecting element 3, it is important that the holder 37 and the slide element 27 cannot be changed in their position relative to each other.

The third connecting element drive 33 is configured to drive a drive bevel gear 38, which is attached to a non-visible drive shaft of the connecting element drive 33. The drive bevel gear 38 is in engagement with a driven bevel gear 39, which is arranged on an output shaft 40, which is rotatably mounted in the slide element 27. The output shaft 40 can be directly connected to the end effector 6 and be configured as the pivot joint axis of rotation 31.

According to any of the preceding embodiments, a sterile drape may be used to cover the resection device completely or partially. The sterile drape may also comprise a cover or be formed as a cover. The parts that are not covered by the sterile drape may be intended for single use or may be sterilizable.

The resection device 10, 20 according to each of the embodiments can be controlled by a control unit, for example a microcontroller, according to a specification. The control unit can be integrated into the resection device 10, 20 or located outside the working space. The resection device 10, 20 can be in communication with the control unit during the surgical procedure, for example connected by a cable. In addition, a navigation system can be used during the operation, which can also be in communication with the control unit. A screen can also be in communication with the control unit, and various data can be displayed on the screen, for example the operation planning, the current position of the resection device or the progress of the operation.

The target for resection of the at least one bone of the bone arrangement is automatically calculated based on the resection plane, which may be predetermined by an expert, for example by an implant position. The target can comprise a control of the path the tool takes to remove one or more layers of bone material from the bone. Prior to autonomous resection, the proposed path can be checked and adjusted if necessary.

A possible workflow incorporating the resection device is outlined below:
Planning the procedure with a planning software using CT scans:
Attaching the fastening element 4 to the bone arrangement 8 and coupling the resection device 10, 20 using an optional positioning and registration system.
Performing the planned bone resection or bone resections. For example, the bone arrangement 8 can include a plurality of bones, such as a femur 18 and a tibia 28. The resection can be performed on at least one bone of the bone arrangement 8, on a plurality of the bones of the bone arrangement 8, in particular on each of the bones of the bone arrangement 8.

Optionally, the tension of ligaments or tendons can be determined. If necessary, an adjustment incision can be made.

Optionally, a tracking system can be used to monitor the entire procedure.

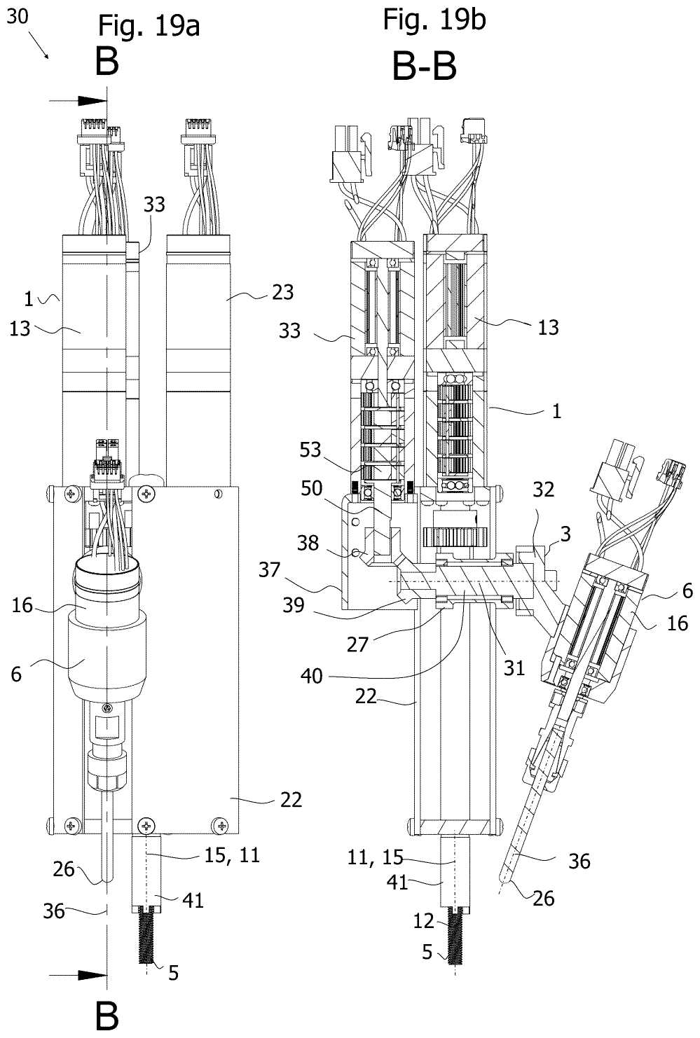

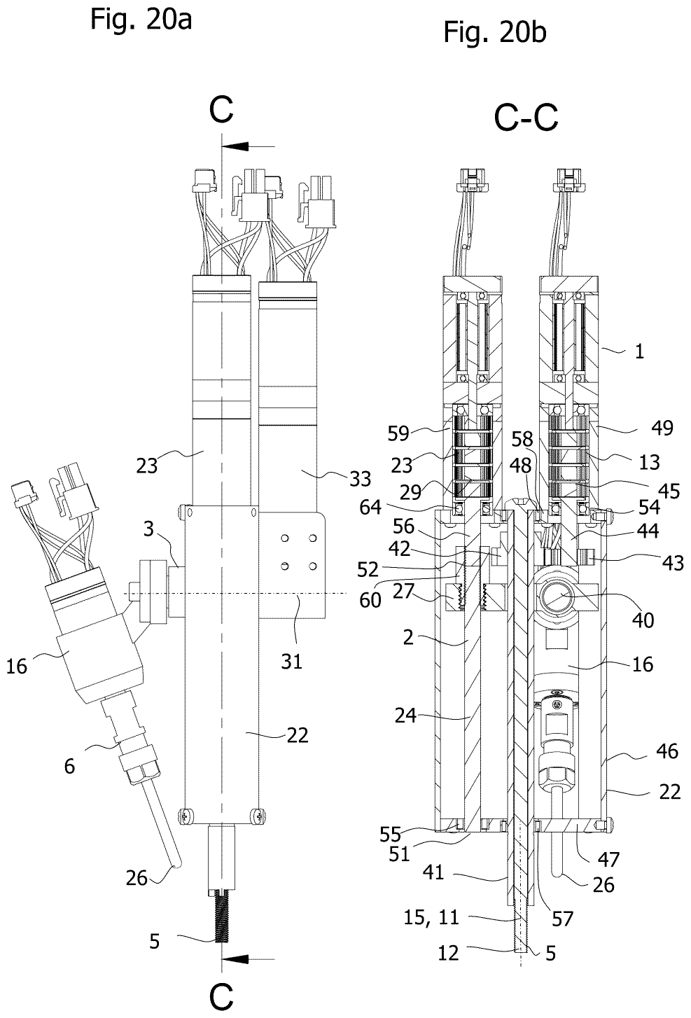

FIG. 19a shows a front view of a resection device 30 according to a third embodiment. FIG. 19b shows a section through the resection device 30 according to FIG. 19a along the section plane represented by the section line B-B. FIG. 20a shows a side view of the resection device 30. FIG. 20b shows a section through the resection device 30 according to FIG. 20a along the section plane represented by the section line C-C. The resection device 30 is configured to be attached to a bone arrangement 8. The same reference symbols have been used for components whose function corresponds to the earlier embodiments. The resection device 30 for resecting at least one bone of the bone arrangement 8 without user interaction comprises a fastening element 4, wherein the fastening element 4 is configured to be rigidly fastened to the bone arrangement 8. The bone arrangement 8 and the fastening element 4 are omitted in the present description for the sake of simplicity; see, for example, FIG. 1.

The resection device 30 further comprises a base element 5, wherein the fastening element includes the base element 5 or the fastening element 4 can be coupled to the base element 5 in such a way that the base element 5 forms a rigid connection with the fastening element 4. The base element 5 includes a base element longitudinal axis 15. The resection device 30 further comprises a rotatable connecting element 1, which is rotatably arranged about the base element 5, wherein the rotatable connecting element 1 is rotatably arranged about the base element longitudinal axis 15, so that the base element longitudinal axis 15 forms an axis of rotation 11 for the rotatable connecting element 1.

According to the present embodiment, the base element 5 comprises a sleeve element 41 shown in section in FIG. 20b, which is firmly connected to a core element 12 of the base element 5. The sleeve element 41 includes an output gear 42, which is also rigidly connected to the sleeve element 41. The sleeve element 41 is in meshing engagement with a drive gear 43 via the output gear 42. The drive gear 43 is connected in a rotationally fixed manner to a drive shaft 44. The drive gear 43 is driven by the first connecting element drive 13. According to the present embodiment, the rotatable connecting element 1 is formed by the drive gear 43 and the first connecting element drive 13. The first connecting element drive 13 may in particular comprise the drive shaft 44 and the drive motor 45. In the present example, the drive motor 45 is designed as an electric motor. When the drive gear 43 is driven by the drive motor 45 via the drive shaft 44, the rotatable connecting element 1 rotates about the axis of rotation 11.

According to the present embodiment, the rotatable connecting element 1 comprises a housing 22. The first connecting element drive 13 is located at least partially in the housing 22. According to the present embodiment, the housing 22 comprises a housing shell element 46, a housing base element 47, and a housing cover element 48, see in particular FIG. 20b. The housing 22 is rotatable relative to the sleeve element 41. The housing 22 is thus configured such that, when the first connecting element drive 13 is actuated, it rotates about the axis of rotation 11 of the base element 5. According to the present embodiment, the housing 22 is thus part of the rotatable connecting element 1. According to the present embodiment, the housing base element 47 and the housing cover element 48 each include a bearing element 57, 58. The housing base element 47 is rotatable relative to the base element 5 by means of the bearing element 57. According to the present embodiment, the bearing element 57 is located on the sleeve element 41. The housing cover element 48 is rotatably mounted relative to the base element 5 by means of the bearing element 58. According to the present embodiment, the bearing element 58 is located on the sleeve element 41.

According to this embodiment, the first connecting element drive 13 comprises a drive housing 49 for the drive motor 45. The drive housing 49 is also connected to the housing 22. In particular, the drive housing 49 can participate in a rotary movement of the housing 22 when the first connecting element drive 13 is actuated. The drive shaft 44 is rotatably mounted in the drive housing 49 by means of the drive shaft bearing element 54.

When the drive motor 45 is started, the drive shaft 44 is set in rotation. The drive shaft 44 sets the drive gear 43 in motion. The drive gear 43 is in meshing engagement with the output gear 42. The output gear is fixedly connected to the sleeve element 41, with the result that the drive gear performs a circular movement around the output gear 42 in the manner of a planetary gear. This circular movement is transmitted to the housing 22. The housing 22 thus rotates with the first connecting element drive 13 about the axis of rotation 11. According to the present embodiment, the housing 22 contains the displaceable connecting element 2. The housing 22 is thus rotatable with the first connecting element drive 13 about the axis of rotation 11, with the displaceable connecting element 2 also performing this rotational movement. In other words, the displaceable connecting element 2 is coupled to the rotatable connecting element 1 via the housing 22.

The rotatable connecting element 1 can be rotated clockwise or counterclockwise. The direction of rotation of the rotatable connecting element 1 can be changed. In particular, the drive shaft 44 can be rotated clockwise or counterclockwise if the drive motor 45 can be operated in both directions of rotation.

The resection device 30 further comprises a displaceable connecting element 2, wherein the displaceable connecting element 2 can perform a linear movement parallel to the axis of rotation 11 relative to the base element 5. According to the present embodiment, the displaceable connecting element 2 is at least partially accommodated in the housing 22. According to this embodiment, the displaceable connecting element 2 comprises the second connecting element drive 23. The spindle element 24 is driven by the second connecting element drive 23. A slide element 27 can be moved with the spindle element 24. The second connecting element drive 23 comprises a spindle element drive motor 29, which is configured as an electric motor, and a drive shaft 56.

According to the present embodiment, the spindle element 24 has a first spindle element end 51, which is rotatably mounted in the housing base element 47 of the housing 22. For this purpose, according to the present embodiment, a bearing element 55 is provided in the housing base element 47.

The spindle element 24 has a second spindle element end 52 which can be coupled to the second connecting element drive 23 via a coupling element 60. The second connecting element drive 23 can in particular comprise a drive shaft 56 and a spindle element drive motor 29. According to this embodiment, the second connecting element drive 23 includes a drive housing 59 for the drive motor 29. The drive housing 49 is also connected to the housing 22. In particular, the drive housing 59 can participate in a rotary movement of the housing 22 when the first connecting element drive 13 is actuated. The drive shaft 56 is rotatably mounted in the drive housing 59 by means of the drive shaft bearing element 64.

The spindle element 24 can rotate clockwise or counterclockwise. The direction of rotation of the spindle element 24 can be changed. In particular, the drive shaft 56 can rotate clockwise or counterclockwise if the drive motor 29 can be operated in both directions of rotation.

The resection device 30 further comprises a connecting element 3, wherein the connecting element 3 is connected to one of the rotatable or displaceable connecting elements 1, 2 via a pivot joint 32. The connecting element 3 is configured as a processing device for performing bone resection. The pivot joint 32 includes a pivot joint axis of rotation 31, whereby the connecting element 3 can rotate about the pivot joint axis of rotation 31, whereby the pivot joint axis of rotation 31 is arranged perpendicular to the axis of rotation 11 according to this embodiment. The connecting element 3 includes an end effector 6 for bone resection. The connecting element 3 can form a unit with the end effector 6. The end effector 6 can also be coupled to the connecting element 3. For example, different end effectors 6 can be attached to the connecting element 3 as required. Of course, according to an embodiment not shown, the connecting element marked with reference numeral 2 can be configured as the rotatable connecting element and the connecting element marked with reference numeral 1 can be configured as the displaceable connecting element.

The end effector 6 includes the tool 26, in particular a bone resection tool, which may be, for example, a milling device or a sawing element. The end effector 6 can include an end effector rotation axis 36 about which the tool 26 can rotate. The end effector rotation axis 36 can coincide with the longitudinal axis of the end effector 6. In addition, the end effector 6 can have an end effector drive 16.

The rotatable connecting element 1, the displaceable connecting element 2, and the connecting element 3 can be driven independently of one another by means of corresponding connecting element drives 13, 23, 33. In particular, the rotatable connecting element 1 can be driven by a first connecting element drive 13, the displaceable connecting element 2 can be driven by a second connecting element drive 23, and the connecting element 3 can be driven by a third connecting element drive 33. In particular, at least one of the connecting element drives 13, 23, 33 is housed in a compact block that can be removed from the housing. According to the present embodiment, the rotatable connecting element 1 is configured as a housing 22 and the displaceable connecting element 2 is arranged on the housing 22. According to the present embodiment, a common housing 22 is formed by the rotatable connecting element 1 and the displaceable connecting element 2. By means of the first connecting element 1, the housing 22 and the displaceable connecting element rotatably mounted in the housing 22 are rotated via the first connecting element drive.

According to this embodiment, the first connecting element drive 13 and the second connecting element drive 23 are arranged in or on a common housing 22. The first connecting element drive 1 rotates the housing 22 of the resection device 10 about the longitudinal axis 15 of the base element, which forms the axis of rotation 11. According to this embodiment, the rotatable connecting element 1 comprises the housing 22. According to this embodiment, the displaceable connecting element 2 is attached to the housing 22 in such a way that, when the rotatable connecting element 1 rotates, the displaceable connecting element 2 is also rotated. The translational movement of the displaceable connecting element 2 can occur simultaneously or with a time delay in relation to the rotation of the rotatable connecting element 1. It is therefore possible for the rotatable and displaceable connecting elements 1 and 2 to be actuated simultaneously.

It is also possible that at a first point in time only a rotation of the rotatable connecting element 1 takes place and at a second point in time only the displacement of the displaceable connecting element 2 takes place. The first point in time can thus differ from the second point in time. The first and second points in time can also coincide if the first connecting element drive 13 and the second connecting element drive 23 are in operation at the same time. The rotation of the connecting element 3 can also take place simultaneously or with a time delay in relation to the rotation of the rotatable connecting element 1 or the translational movement of the displaceable connecting element 2. It is also possible that at a first point in time only a rotation of the rotatable connecting element 1 takes place, at a second point in time only the displacement of the displaceable connecting element 2 takes place, and at a third point in time only the rotation or displacement of the connecting element 3 takes place. The first point in time can thus differ from at least one of the second or third points in time. The first, second, and third points in time can also coincide if the first connecting element drive 13, the second connecting element drive 23, and the third connecting element drive 33 are in operation at the same time.

According to the embodiment shown in FIG. 19b, the slide element 27 includes a holder 37 for the third connecting element drive 33. The holder 37 can also comprise a housing element. According to this embodiment, the slide element 27 and the holder 37 are configured as a single component. However, according to a non-illustrated embodiment, it is also possible for the holder 37 and the slide element 27 to be configured as two or more components which are, however, rigidly connected to one another, for example by a screw connection, a welded connection or another connecting means. For the connecting element 3 to function, it is essential that the holder 37 and the slide element 27 cannot change their position relative to each other.

The third connecting element drive 33 is configured to drive a drive bevel gear 38, which is attached to a drive shaft 50 of the connecting element drive 33. The connecting element drive 33 comprises a drive motor 53, the drive shaft 50 and the drive bevel gear 38. The drive shaft 50 connects the drive motor 53 to the drive bevel gear 38. The drive bevel gear 38 is in engagement with a driven bevel gear 39, which is arranged on a driven shaft 40 which is rotatably mounted in the slide element 27. The output shaft 40 can be connected directly to the end effector 6 and includes the pivot joint axis of rotation 31.

According to this embodiment, the resection device has at least 3 degrees of freedom (DOF). The first DOF comprises rotation of the rotatable connecting element 1 about the axis of rotation 11, which corresponds to the longitudinal axis 15 of the base element 5. The second DOF comprises a translational movement of the displaceable connecting element 2 in the direction of the axis of rotation 11 of the rotatable connecting element 1, in particular parallel to the axis of rotation 11. According to the present embodiment, the third DOF comprises a rotation of the connecting element 3 about a pivot joint axis of rotation 31. The pivot joint axis of rotation can be arranged in particular perpendicular to the axis of rotation 11 of the rotatable connecting element 1, or it can also be arranged at an angle of 60 degrees to 90 degrees inclusive to the axis of rotation 11, which is not shown in the drawings.

It is obvious to a person skilled in the art that many further variants in addition to the systems or process variants described are possible without deviating from the inventive concept. The subject matter of the invention is thus not limited by the preceding description and is determined by the scope of protection defined by the claims. For the interpretation of the claims or the description, the broadest possible reading of the claims is decisive. In particular, the terms “comprising” or “including” are to be interpreted as referring to elements, components, or steps in a non-exclusive meaning, thereby indicating that the elements, components, or steps may be present or used, that they may be combined with other elements, components or steps not explicitly mentioned. When the claims refer to an element or component from a group which may consist of A, B, C to N elements or components, this formulation is to be interpreted as requiring only one element of this group, and not a combination of A and N, B and N or any other combination of two or more elements or components of this group.

What is claimed is:

1. A resection device for resecting at least one bone of a bone arrangement without user interaction, comprising:
a fastening element, the fastening element being configured to be rigidly fastened to the bone arrangement, and
a base element, wherein the fastening element includes the base element or the fastening element can be coupled to the base element such that the base element forms a rigid connection with the fastening element, wherein the base element includes a base element longitudinal axis, and
a rotatable connecting element, which is arranged rotatably about the base element, wherein the rotatable connecting element is arranged rotatably about the base element longitudinal axis, such that the base element longitudinal axis forms an axis of rotation for the rotatable connecting element, wherein the rotatable connecting element includes a first connecting element drive, and
a displaceable connecting element, wherein the displaceable connecting element includes a second connecting element drive, wherein the displaceable connecting element is configured to execute a linear movement along or parallel to the base element longitudinal axis relative to the base element, and
a connecting element, wherein the connecting element includes a third connecting element drive, wherein the connecting element is connected to one of the rotatable or displaceable connecting elements via a pivot joint, wherein the pivot joint includes a pivot joint axis of rotation, wherein the connecting element is rotatable about the pivot joint axis of rotation,
wherein the connecting element includes an end effector for bone resection.

2. The resection device of claim 1, wherein the pivot joint axis of rotation is arranged at an angle of 60 degrees up to and including 90 degrees to the axis of rotation.

3. The resection device of claim 1, wherein the fastening element comprises a connector piece for forming a rigid connection of a plurality of bones of the bone arrangement, wherein the connector piece comprises a fastening means for fastening to the fastening element or to the bone arrangement, wherein the fastening means can comprise at least one fastening element from the group consisting of pin elements, screw elements and clamp elements.

4. The resection device of claim 1, wherein a soft tissue protection element is provided.

5. The resection device of claim 1, wherein the rotatable connecting element, the displaceable connecting element and the connecting element are drivable independently of each other by means of corresponding connecting element drives.

6. The resection device of claim 5, wherein at least one of the connecting element drives is housed in a compact, removable block.

7. The resection device of claim 1, wherein the fastening element comprises a plurality of positions for fastening the base element, wherein the plurality of positions can be adjustable by means of an adjustable coupling element.

8. The resection device of claim 1, wherein the fastening element is configured as a fastening means from the group consisting of an intramedullary rod, a modified intramedullary rod, a bone nail, a bone clamp, a bone screw, and a holder fastened with pin elements.

9. The resection device of claim 1, wherein the end effector comprises a tool selected from the group consisting of a milling device, a sawing device, and a drilling device, wherein the tool can comprise a ball end mill.

10. A resection device for resecting at least one bone of a bone arrangement without user interaction, comprising:
a fastening element, wherein the fastening element is configured to be rigidly fastened to the bone arrangement,
a base element, wherein the fastening element includes the base element or the fastening element can be coupled to the base element in such a way that the base element forms a rigid connection with the fastening element, wherein the base element includes a base element longitudinal axis, and
a rotatable connecting element, which is arranged rotatably about the base element, wherein the rotatable connecting element is arranged rotatably about the base element longitudinal axis, so that the base element longitudinal axis forms an axis of rotation for the rotatable connecting element, wherein the rotatable connecting element includes a first connecting element drive, and
a displaceable connecting element, wherein the displaceable connecting element is configured to execute a linear movement along the base element longitudinal axis relative to the base element, wherein the displaceable connecting element includes a second connecting element drive, and
a connecting element, wherein the connecting element includes a third connecting element drive, wherein the connecting element is connected to one of the rotatable or displaceable connecting elements via a guide rail element, wherein the guide rail element includes a rail element axis, wherein the connecting element is displaceable along the rail element axis,
wherein the connecting element includes an end effector for bone resection.

11. The resection device of claim 10, wherein the rail element axis is arranged at an angle of 60 degrees up to and including 90 degrees to the axis of rotation.

12. The resection device of claim 10, wherein the fastening element comprises a connector piece for forming a rigid connection of a plurality of bones of the bone arrangement.

13. The resection device of claim 12, wherein the connector piece comprises a fastening means for fastening to the fastening element or to the bone arrangement, wherein the fastening means comprises at least one fastening element from the group consisting of pin elements, screw elements and clamp elements.

14. The resection device of claim 10, wherein a soft tissue protection element is provided.

15. The resection device of claim 10, wherein the rotatable connecting element, the displaceable connecting element and the connecting element are drivable independently of each other by means of corresponding connecting element drives.

16. The resection device of claim 15, wherein at least one of the connecting element drives is housed in a compact, removable block.

17. The resection device of claim 10, wherein the fastening element comprises a plurality of positions for fastening the base element.

18. The resection device of claim 17, wherein the plurality of positions are adjustable by means of an adjustable coupling element.

19. The resection device of claim 10, wherein the fastening element is configured as a fastening means from the group consisting of an intramedullary rod, a modified intramedullary rod, a bone nail, a bone clamp, a bone screw, and a holder fastened with pin elements.

20. The resection device of claim 10, wherein the end effector comprises a tool selected from the group consisting of a milling device, a sawing device, and a drilling device, wherein the tool can comprise a ball end mill.
